(19) United States
(12) Patent Application Publication
(10) Pub. No.: US 2003/0113357 A1
(43) Pub. Date: Jun. 19, 2003

(54) Skin Care Compositions

(75) Inventors: Michael David Bell, Crowthorne (GB); Russell Philip Elliott, Egham (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/302,032

(22) Filed: Nov. 22, 2002

Related U.S. Application Data
(63) Continuation of application No. PCT/US01/15596, filed on May 14, 2001.

(30) Foreign Application Priority Data
May 23, 2000 (GB) 00112565.8
Sep. 22, 2000 (GB) 0023336.1
Oct. 5, 2000 (GB) 0024372.5

(51) Int. Cl. A61K 7/00

(52) U.S. Cl. 424/401

(57) ABSTRACT

The present invention relates to a topical cosmetic composition with an aqueous continuous phase, in the form of a lotion, cream or gel, comprising:

i) from 0.01% to 25%, by weight, of an organic particulate material having a median particle size of from 0.5 to 30 μm;

ii) greater than 8%, by weight, of a humectant selected from glycerine, polyalkylene glycol, urea, D or DL panthenol, hexane-1, 2, 6-triol, guanidine or its derivatives, alkylene glycols such as butylene glycol and propylene glycol, and mixtures thereof;

iii) from 2% to 20%, by weight, organic sunscreen.

for providing highly efficacious levels of moisturisation and UV protection while maintaining optimum skin feel. Preferred embodiments are oil-in-water emulsions wherein the particulate material has been pre-dispersed within an organosiloxane oil to provide for desired non tacky skin feel.

### CROSS REFERENCE TO RELATED APPLICATIONS

This application is a continuation of International Application No. PCT/US/01/15596, filed May 14, 2002.

### FIELD OF THE INVENTION

The present invention relates to topical compositions with an aqueous continuous phase suitable for providing high levels of moisturisation within a product which has highly acceptable skin feel properties. More particularly, the invention relates to topical compositions which, in addition to moisturisation, also provide protection against the harmful effects of ultraviolet radiation, while maintaining optimum skin feel. The compositions of the present invention are especially useful for the treatment of the skin on the face and hands and also on other areas of the body.

### BACKGROUND

It has long been a feature of many types of topical compositions, including skin care compositions, that they comprise ingredients with moisturisation, skin regulating and UV protection benefits. Such compositions are used to both improve the skin hydration levels and to protect the skin from the harmful and damaging effects of ultraviolet radiation. In today's environments, when consumers are increasingly exposed to very skin drying conditions, such as air conditioned buildings, and where UV radiation is becoming more harmful as a result of the depletion of the ozone layer, there is an growing desire for skin care products, especially facial products, which protect against, and repair the damaged from, these harmful daily assaults.

Conversely, such skin benefit agents, especially organic sunscreens and water soluble humectants and vitamins, can be highly tacky in their skin feel with the result that compositions containing significant levels of these ingredients have highly undesirable skin feel properties including excessive greasiness, stickiness and application drag. In addition, when these high levels of humectants are combined with thickeners the tackiness can be exacerbated. Thus, until now, there has been a trade off between formulating topical compositions with efficacious levels of said benefit imparting ingredients whilst simultaneously minimising the levels of these same ingredients so that the final product has acceptable skin feel properties for the consumer. Net, to date, in order to achieve acceptable skin feel properties, topical compositions have tended to comprise sub-optimal levels of the efficacious, high tack ingredients resulting in multiple applications over an extended period being required to achieve desired skin benefits. Since ingredients with the worst tack properties are often water soluble this problem is exacerbated in topical compositions with an aqueous continuous phase. This is because the ingredients with the highly tacky properties, when present or solubilised within the aqueous phase, come into high levels of contact with the skin upon application. It is therefore desirable to provide a topical composition with an aqueous continuous phase which comprises efficacious levels of ingredients with skin benefits, particularly moisturisation and UV protection benefits, whilst at the same time having consumer acceptable skin feel properties.

To date the inclusion of particulate materials, including both organic and inorganic particles, have been reported in skin care compositions to affect the texture of both the skin and the composition itself. See, for example, ‘Quantification of the Soft-Focus Effect’, Cosmetics & Toiletries, Vol. 111, July 1996, pp. 57-61, which discloses that skin lines can be physically filled with reflective substances such as TiO₂. U.S. Pat. No. 4,892,726, issued to Toshiba Silicone Co. Ltd, describes the use of polymethylsilsesquioxane powders in makeup and cosmetic compositions which are smooth upon application and impart natural colour. These particles are now available, in a variety of particle size grades, from Toshiba Silicone Co. Ltd under the name Tospearl®. WO 00/24372 describes topical compositions comprising 0.5% to 25% of a particulate material having a refractive index from 1.35 to 1.6 and a particle size from 2 μm to 30 μm, in conjunction with other appearance improving actives such as vitamin B3 compounds and retinoids, for imparting visual improvement to the skin. The particles disclosed therein similarly include Tospearl® 145A which has a median particle size of about 4.5 μm. Similarly, copending application PCT/US99/04748 discloses topical compositions comprising 0.1% to 10% organic particulate material, again with a refractive index from 1.3 to 1.7, which are suitable for imparting an essentially immediate improvement in skin feel and appearance. EP-A-692,242 discloses the use of hollow, deformable particles of a size of from 1 to 250 μm, most preferably 18 μm for reducing the sticky feel of essentially anhydrous compositions which are rich in fatty substances; EP-A-908,175 discloses stable cosmetic compositions comprising at least one fatty phase, cross linked elastomeric organopolysiloxanes and high levels of organosiloxane particles; and EP-A-790,055 discloses the use of partially cross linked elastomeric organosiloxane polymers, in combination with a fatty phase and preferably enclosed within a fatty phase, for preparing compositions which provide a matt appearance to the skin. These applications disclose a variety of cosmetic compositions with improved skin feel and which additionally have improved skin appearance properties. However, the applications do not disclose aqueous compositions with highly efficacious levels of moisturisation and UV protection benefit agents.

The compositions and disclosures of the prior art provide useful advances in the art of cosmetic skin treatment and the use of organic particulates in conjunction with skin benefit agents. However, they do not adequately teach how to provide both efficacious levels of skin moisturisation and UV protection ingredients in an aqueous based topical composition which additionally has consumer acceptable skin feel properties.

Surprisingly it has now been found that the high tack negatives of topical skin care compositions with an aqueous continuous phase, which also contain highly efficacious levels of moisturisation and UV protection ingredients, can be reduced by additionally comprising one or more types of organic particulate, with a defined particle size and critical surface tension, within the composition. Importantly, the improvements of the skin feel properties are so considerable that high levels of moisturisation in conjunction with organically derived UV protection, can be achieved within a formulation. Previously such combinations would not have been possible due to the unacceptable tackiness. Additionally it has also been found that if the organic particulate is pre-mixed in a low viscosity organosiloxane oil the improvement in skin feel achieved is even greater.

Without wishing to be bound by theory it is believed that inclusion of the particulate materials contributes to minimising friction on product application. Upon product application the particles locate themselves at the product residue/air interface, sitting proud of the product residue surface. Thus the contact of the skin with the product residue is minimised and the skin feel is improved. In order to ensure that the particulates are delivered in the most efficient way to the skin surface during application it is preferred that the particulate is premixed in a carrier oil. The particulates are completely wetted out by the carrier oil and the low viscosity, and therefore the high mobility, of the pre-mix ensures that the particles are efficiently spread over the skin surface on product application.

It is an object of the present invention to provide topical compositions with an aqueous continuous phase suitable for imparting high levels of moisturisation and UV protection to the skin. It is another object of the present invention to provide topical compositions containing a particulate material e.g., polymethylsilsesquioxane, which off sets the tackiness of the formulations to deliver desirable skin feel properties. It is another object of the present invention to provide compositions which are useful for delivering desired levels of moisturisation and UV protection without the need for multiple applications.

These, and other objects of this invention, will become apparent in light of the following disclosure.

### SUMMARY OF THE INVENTION

The present invention relates to topical cosmetic composition with an aqueous continuous phase, in the form of a lotion, cream or gel, comprising:

i) from 0.01% to 25%, by weight, of an organic particulate material having a median particle size of from 0.5 to 30 μm;

ii) greater than 8%, by weight, of a humectant selected from glycerine, polyalkylene glycol, urea, D or DL panthenol, hexane-1, 2, 6-triol, guanidine or its derivatives, alkylene glycols such as butylene glycol and propylene glycol, and mixtures thereof;

iii) from 2% to 20%, by weight, organic sunscreen.

The compositions are useful for imparting highly efficacious levels of moisturisation and UV protection whilst maintaining acceptable skin feel properties.

This invention further relates to manufacturing methods for enhancing the skin feel benefits and cosmetic methods of delivering the moisturisation and UV protections benefits by topical application of the subject compositions.

### DETAILED DESCRIPTION OF THE INVENTION

All percentages and ratios used herein are by weight of the total composition and all measurements made are at 25° C., unless otherwise designated. Unless otherwise indicated all percentages, ratios and levels of ingredients referred to herein are based on the actual amount of the ingredient, and do not include solvent, fillers or other materials which may be combined with the ingredient in commercially available products.

All publications cited herein are hereby incorporated by reference in their entirety, unless otherwise indicated.

The term “dermatologically-acceptable,” as used herein, means that the compositions, or components thereof, are suitable for use in contact with human skin without undue toxicity, incompatibility, instability, allergic response, and the like.

The term “safe and effective amount” as used herein means an amount of a compound, component, or composition sufficient to significantly induce a positive benefit, preferably a positive skin appearance or feel benefit, including independently the benefits disclosed herein, but low enough to avoid serious side effects, i.e. to provide a reasonable benefit to risk ratio, within the scope of sound medical judgement.

Active and other ingredients useful herein may be categorised or described herein by their cosmetic and/or therapeutic benefit or their postulated mode of action. However, it is to be understood that the active and other ingredients useful herein can in some instances provide more than one cosmetic and/or therapeutic benefit or operate via more than one mode of action. Therefore, classifications herein are made for the sake of convenience and are not intended to limit an ingredient to the particularly stated application or applications listed.

The elements of these compositions are described in more detail below.

### Essential Elements of the Composition

Organic Particulate Material

The composition comprises from about 0.01% to about 25%, preferably from about 0.1% to about 15%, more preferably from about 0.2% to about 5%, of organic particulate. It is highly preferred that the organic particulate has a median particle size of from about 0.5 to about 30 μm, preferably from about 0.5 to about 15 μm, more preferably from about 0.7 to about 10 μm and even more preferably from about 1 to about 7.5 μm. The median particle size is refers to the particulate material in the neat form i.e. in the essentially pure, powder form. Particular methods of measuring particle size may, however, require the particulate material to be dispersed in an inert carrier, such as a pure oil, in order to measure the particle size distribution. Median particle size can be determined by any suitable method known in the art, such as by using coulter-counter equipment or the ASTM Designation E20-85 “Standard Practice for Particle Size Analysis of Particulate Substances in the Range of 0.2 to 75 Micrometers by Optical Microscopy”, ASTM Volume 14.02, 1993. It is preferred that more than 50%, preferably more than 60%, more preferably more than 70% of particles fall within the size ranges prescribed for the respective median values.

Preferred particles are free-flowing, solid materials. By solid it is meant that the particles are preferably non elastomeric and non deformable. Particles are selected from organosilicone polymers, polyethylene, polyacrylonitrile, polyacrylic acid or derivatives thereof, polymethacrylic acid, polystyrene, polytetrafluoroethylene (PTFE), poly(vinylidene chloride), polyamide and mixtures thereof. More preferably organic particulate material is selected from organosilicone polymers and mixtures thereof, and even more preferably is polymethylsilsesquioxane. Copolymers derived from monomers of the afore-mentioned materials can also be used. Suitable commercially available examples of the polymethylsilsesquioxane organic particulate material are Tospearl® 145 which has a median particle size of about 4.5 μm and Tospearl® 1320 which has a median particle size of 12□m. A further representative commercially available example is Fluoropure 100C from Shamrock Inc, which is a polytetrafluoroethylene organic particulate material. Another representative commercially available example is EA-209® from Kobo which is an ethylene/acrylic acid copolymer having a median particle size of about 10 μm.

The organic particulate material preferably has a critical surface tension of less than 50 mN/m, preferably less than 40 mN/m and more preferably less than 35 mN/m. The critical surface tension is taken to mean that as defined by Drew and Myers in “Surfaces, Interfaces and Colloids, Principles and Applications” 1991 Ch 17, pp 364-369, (VCH Publishers Inc, New York) and is measured, in the absence of doubt, by contact angle measurements of a series of related liquids with different surface tensions as also outlined in the aforementioned reference.

Humectants

Compositions of the present invention comprise from about 8% to about 30%, preferably from about 8.5% to about 20% and more preferably from about 9% to about 15%, by weight, humectant. Preferred humectants include, but are not limited to, compounds selected from glycerine, polyoxyalkylene glycol, urea, D or DL panthenol, hexane-1, 2, 6,-triol, guanidine or its derivatives, alkylene glycols such as butylene glycol or propylene glycol. Highly preferred humectants are glycerine, urea and mixtures thereof. The above listed compounds may be incorporated singly or in combination. The aforementioned compounds may give rise to high tack levels, especially when used at the preferred levels of greater than 8%. It has been found, however, that this tacky feel can be effectively offset by using the humectants in combination with the organic particulates, as described by the present invention.

Organic Sunscreens

Compositions of the present invention comprise an organic sunscreen. Suitable sunscreens can have UVA absorbing properties, UVB absorbing properties or a mixture thereof. The exact amount of the sunscreen active will vary depending upon the desired Sun Protection Factor, i.e., the “SPF” of the composition as well as, the desired level of UVA protection. The compositions of the present invention preferably comprise an SPF of at least 10, preferably at least 15. (SPF is a commonly used measure of photoprotection of a sunscreen against erythema. The SPF is defined as a ratio of the ultraviolet energy required to produce minimal erythema on protected skin to that required to products the same minimal erythema on unprotected skin in the same individual. See Federal Register, 43, No 166, pp. 38206-38269, Aug. 25, 1978). Compositions of the present invention comprise from about 2% to about 20%, more typically from about 4% to about 14%, by weight, of organic sunscreen. Suitable sunscreens include, but are not limited to, those found in the CTFA International Cosmetic Ingredient Dictionary and Handbook, 7^{th} edition, volume 2 pp. 1672, edited by Wenninger and McEwen (The Cosmetic, Toiletry, and Fragrance Association, Inc., Washington, D.C., 1997).

The compositions of the present invention preferably comprise a UVA absorbing sunscreen actives which absorb UV radiation having a wavelength of from about 320 nm to about 400 nm. Suitable UVA absorbing sunscreen actives are selected from dibenzoylmethane derivatives, anthranilate derivatives such as methylanthranilate and homomethyl, 1-N-acetylanthranilate, and mixtures thereof. Examples of dibenzoylmethane sunscreen actives are described in U.S. Pat. No. 4,387,089 issued to Depolo; and in Sunscreens: Development, Evaluation, and Regulatory Aspects edited by N. J. Lowe and N. A. Shaath, Marcel Dekker, Inc (1990). The UVA absorbing sunscreen active is preferably present in an amount to provide broad spectrum UVA protection either independently, or in combination with, other UV protective actives which may be present in the composition.

Preferred UVA sunscreen actives are dibenzoylmethane sunscreen actives and their derivatives. They include, but are not limited to, those selected from 2-methyldibenzoylmethane, 4-methyidibenzoylmethane, 4-isopropyldibenzoylmethane, 4-tert-butyldibenzoylmethane, 2, 4-dimethyldibenzoylmethane, 2, 5-dimethyidibenzoylmethane, 4, 4′-diisopropylbenzoylmethane, 4-(1, 1-dimethylethyl)-4′-methoxydibenzoylmethane, 2-methyl-5-isopropyl-4′-methoxydibenzoylmethane, 2-methyl-5-tert-butyl-4′-methoxy-dibenzoylmethane, 2, 4-dimethyl-4′-methoxydibenzoylmethane, 2, 6-dimethyl-4′-tert-butyl-4′methoxydibenzoylmethane, and mixtures thereof. Preferred dibenzoyl sunscreen actives include those selected from 4-(1, 1-dimethylethyl)-4′-methoxydibenzoylmethane, 4-isopropyldibenzoylmethane, and mixtures thereof. A more preferred sunscreen active is 4-(1, 1-dimethylethyl)-4′-methoxydibenzoylmethane.

The sunscreen active 4-(1, 1-dimethylethyl)-4′-methoxydibenzoylmethane, which is also known as butyl methoxydibenzoylmethane or Avobenzone, is commercially available under the names of Parsol® 1789 from Givaudan Roure (International) S. A. (Basel, Switzerland) and Eusolex® 9020 from Merck & Co., Inc (Whitehouse Station, N.J.). The sunscreen 4-isoproplydibenzoylmethane, which is also known as isopropyidibenzoylmethane, is commercially available from Merck under the name of Eusolex® 8020.

The compositions of the present invention preferably further comprise a UVB sunscreen active which absorbs UV radiation having a wavelength of from about 290 nm to abut 320 nm. The compositions comprise an amount of the UVB sunscreen active which is safe and effective to provide UVB protection either independently, or in combination with, other UV protective actives which may be present in the compositions. The compositions preferably comprise from about 0.1% to abut 16%, more preferably from about 0.1% to about 12%, and most preferably from about 0.5% to about 8% by weight, of UVB absorbing organic sunscreen.

A wide variety of UVB sunscreen actives are suitable for use herein. Nonlimiting examples of such organic sunscreen actives are described in U.S. Pat. No. 5,087,372 issued Feb. 11, 1992 to Haffey et al.; and U.S. Pat. Nos. 5,073,371 and 5,073,372 both issued on Dec. 17, 1991 to Turner et al.. Preferred UVB sunscreen actives are selected from 2-ethylhexyl-2-cyano-3, 3-diphenylacrylate (referred to as octocrylene), 2-phenyl-benzimidazole-5-sulphonic acid (PBSA), cinnamates and their derivatives such as 2-ethylhexyl-p-methoxycinnamate and octyl-p-methoxycinnamate, TEA salicylate, octyldimethyl PABA, camphor derivatives and their derivatives, and mixtures thereof. Preferred organic sunscreen actives are 2-ethylhexyl-2-cyano-3, 3-diphenylacrylate (referred to as octocrylene), 2-phenyl-benzimidazole-5-sulphonic acid (PBSA), octyl-p-methoxycinnamate, and mixtures thereof. Salt and acid neutralised forms of the acidic sunscreens are also useful herein. When organic sunscreen salts, such as PBSA, are used within compositions of the present invention they can disrupt the action of the thickener with the result that the final product may have sub optimal rheology. This can be countered by the addition of higher levels of thickener, fatty alcohols or nonionic surfactants such that the rheology of the final product returns to the desired level.

An agent may also be added to any of the compositions useful in the present invention to stabilise the UVA sunscreen to prevent it from photo-degrading on exposure to UV radiation and thereby maintaining its UVA protection efficacy. A wide range of compounds have been cited as providing these stabilising properties and should be chosen to compliment both the UVA sunscreen and the composition as a whole. Suitable stabilising agents include, but are not limited to, those described in U.S. Pat. Nos. 5,972,316; 5,968,485; 5,935,556; 5,827,508 and Patent WO 00/06110. Preferred examples of stabilising agents for use in the present invention include 2-ethylhexyl-2-cyano-3, 3-diphenylacrylate (referred to as octocrylene), ethyl-2-cyano-3, 3-diphenylacrylate, 2-ethylhexyl-3, 3-diphenylacrylate, ethyl-3, 3-bis(4-methoxyphenyl)acrylate, and mixtures thereof. 2-ethylhexyl-2-cyano-3, 3-diphenylacrylate is most preferred.

An agent may also be added to any of the compositions useful in the present invention to improve the skin substantivity of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157, Brock, issued May 5, 1987.

Balance of the Composition

The compositions described herein are aqueous, preferably oil-in-water emulsions, and can contain a variety of other optional components suitable for rendering such compositions more cosmetically, aesthetically, or otherwise, acceptable or to provide them with additional usage benefits, provided that such optional components are physically and chemically compatible with the essential components, and method of preparation, described herein and do not unduly impair stability, efficacy or other use benefits associated with the compositions of the present invention. Optional components may be dispersed, dissolved or the like in the carrier of the present compositions.

Such conventional optional ingredients are well known to those skilled in the art. These include, but are not limited to, emollients, oil absorbents, antimicrobial agents, binders, buffering agents, denaturants, cosmetic astringents, external analgesics, film formers, humectants, opacifying agents, perfumes, pigments, skin soothing and healing agents, preservatives, propellants, skin penetration enhancers, solvents, suspending agents, emulsifiers, cleansing agents, thickening agents, solubilising agents, waxes, inorganic sunblocks, sunless tanning agents, antioxidants and/or radical scavengers, chelating agents, anti-acne agents, anti-inflammatory agents, desquamation agents/exfoliants, organic hydroxy acids, vitamins, natural extracts and inorganic particulates such as silica and boron nitride. Nonexclusive examples of such materials are described in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); and in The Handbook of Cosmetic Science and Technology, 1st Ed.. Knowlton & Pearce (Elsevier 1993) can also be used in the present invention.

Preferred compositions have an apparent viscosity of from about 5,000 to about 200,000 mPa.s (centipoise). For example, preferred lotions have an apparent viscosity of from about 10,000 to about 40,000 mPa.s; preferred creams have an apparent viscosity of from about 30,000 to about 160,000 mPa.s. Apparent viscosity can be determined using a Brookfield DVII RV viscometer, spindle TD, at 5 rpm, or the equivalent thereof. The viscosity is determined on the composition after the composition has been allowed to stabilise following its preparation, generally at least 24 hours under conditions of 25° C. and ambient pressure after preparation of the composition. Apparent viscosity is measured with the composition at a temperature of 25° C., after 30 seconds spindle rotation.

The compositions of the present invention are usually formulated to have a pH of 9.5 or below and in general have a pH in the range from about 4.5 to about 9, more preferably from about 5 to about 8.5. Some compositions, particularly those comprising an additional active such as salicylic acid, require a lower pH in order for the additional active to be fully efficacious. These compositions are usually formulated to have a pH of from about 2.5 to about 5, more preferably from about 2.7 to about 4.

Carrier

The compositions of the present invention preferably comprise a safe and effective amount of a dermatologically acceptable carrier, suitable for topical application to the skin within which the essential materials and optional other materials are incorporated to enable the essential materials and optional components to be delivered to the skin at an appropriate concentration. The carrier can thus act as a diluent, dispersant, solvent, or the like for any active ingredients which ensures that they can be applied to, and distributed evenly over, the selected target at an appropriate concentration.

Compositions of the present invention comprise an aqueous continuous phase and preferably comprise an oil-in-water emulsion in which a hydrophobic phase, e.g., a lipid, oil or oily material, is dispersed within the aqueous phase. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. Oil-in-water emulsions typically comprise from about 1% to about 50% (preferably about 1% to about 30%) of the dispersed hydrophobic phase and from about 1% to about 99% (preferably from about 40% to about 90%) of the continuous hydrophilic phase. The emulsion may also comprise a gel network, such as described in G. M. Eccleston, Application of Emulsion Stability Theories to Mobile and Semisolid O/W Emulsions, Cosmetics & Toiletries, Vol. 101, November 1996, pp. 73-92, incorporated herein by reference.

Highly preferred carriers are liquid or semi-solid, such as creams, lotions and gels. Preferably the carrier is in the form of a lotion, cream or a gel, with sufficient thickness, or yield point, to prevent the particles from sedimenting. The carrier can itself be inert or it can possess dermatological benefits of its own. The carrier should also be physically and chemically compatible with the essential components described herein, and should not unduly impair stability, efficacy or other use benefits associated with the compositions of the present invention.

The type of carrier utilised in the present invention depends on the types of product form desired for the composition. The topical compositions useful in the subject invention may be made into a wide variety of product forms such as are known in the art. These include but are not limited to, lotions, creams, gels, sticks, sprays, ointments, pastes and mousses. These product forms may comprise several types of carriers including, but not limited to, solutions, aerosols, emulsions, gels, solids and liposomes.

Preferred carriers contain a dermatologically acceptable, hydrophilic diluent. Suitable hydrophilic diluents include water, organic hydrophilic diluents such as C₁-C₄ monohydric alcohols and low molecular weight glycols and polyols, including propylene glycol, polyethylene glycol (e.g. of MW 200-600), poly-propylene glycol (e.g. of MW 425-2025), glycerol, butylene glycol, butane-1, 2, 4-triol, sorbitol esters, hexane-1, 2, 6-triol, ethanol, iso-propanol, ethoxylated ethers, propoxylated ethers and combinations thereof. The diluent is preferably liquid. Water is an especially preferred diluent. The composition preferably comprises at least about 60% of the hydrophilic diluent.

Emollients

The topical compositions of the subject invention generally comprise from about 1% to about 50%, preferably from about 1.5% to about 15% of a dermatologically acceptable emollient. Emollients tend to lubricate the skin, increase the smoothness and suppleness of the skin, prevent or relieve dryness of the skin, and/or protect the skin. Emollients are typically water-immiscible, oily or waxy materials and emollients with high molecular weights can confer tacky properties to a topical composition. A wide variety of suitable emollients are known and may be used herein. Sagarin, Cosmetics, Science and Technolog, 2nd Edition, Vol. 1, pp. 32-43 (1972), contains numerous examples of materials suitable as an emollient. All emollients discussed in application WO 00/24372 should be considered as suitable for use in the present invention although preferred examples are outlined in further detail below:

i) Straight and branched chain hydrocarbons having from about 7 to about 40 carbon atoms, such as dodecane, squalane, cholesterol, hydrogenated polyisobutylene, isohexadecane and the C₇-C₄₀ isoparaffins, which are C₇-C₄₀ branched hydrocarbons.

ii) C₁-C₃₀ alcohol esters of C₁-C₃₀ carboxylic acids and of C₂-C₃₀ dicarboxylic acids, e.g. isononyl isononanoate, isopropyl myristate, myristyl propionate, isopropyl stearate, isopropyl isostearate, methyl isostearate, behenyl behenate, dioctyl maleate, diisopropyl adipate, and diisopropyl dilinoleate.

iii) C₁-C₃₀ mono- and poly-esters of sugars and related materials. These esters are derived from a sugar or polyol moiety and one or more carboxylic acid moieties. Depending on the constituent acid and sugar, these esters can be in either liquid or solid form at room temperature. Examples include: glucose tetraoleate, the galactose tetraesters of oleic acid, the sorbitol tetraoleate, sucrose tetraoleate, sucrose pentaoleate, sucrose hexaoleate, sucrose heptaoleate, sucrose octaoleate, sorbitol hexaester in which the carboxylic acid ester moieties are palmitoleate and arachidate in a 1:2 molar ratio, and the octaester of sucrose wherein the esterifying carboxylic acid moieties are laurate, linoleate and behenate in a 1:3:4 molar ratio. Other materials include cottonseed oil or soybean oil fatty acid esters of sucrose. Other examples of such materials are described in WO 96/16636, incorporated by reference herein. A particularly preferred material is known by the INCI name sucrose polycottonseedate

iv) Vegetable oils and hydrogenated vegetable oils. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, partially and fully hydrogenated oils from the foregoing sources, and mixtures thereof.

Emulsifiers/Surfactants

Compositions herein preferably contain an emulsifier and/or surfactant, generally to help disperse and suspend the discontinuous phase within the continuous aqueous phase. A surfactant may also be useful if the product is intended for skin cleansing. For convenience hereinafter emulsifiers will be referred to under the term ‘surfactants’, thus ‘surfactant(s)’ will be used to refer to surface active agents whether used as emulsifiers or for other surfactant purposes such as skin cleansing. Known or conventional surfactants can be used in the composition, provided that the selected agent is chemically and physically compatible with essential components of the composition, and provides the desired characteristics. Suitable surfactants include non-silicone derived materials, and mixtures thereof. All surfactants discussed in application WO 00/24372 should be considered as suitable for use in the present invention.

The compositions of the present invention preferably comprise from about 0.05% to about 15% of a surfactant or mixture of surfactants. The exact surfactant or surfactant mixture chosen will depend upon the pH of the composition and the other components present.

Preferred surfactants are nonionic. Among the nonionic surfactants that are useful herein are those that can be broadly defined as condensation products of long chain alcohols, e.g. C₈₋₃₀ alcohols, with sugar or starch polymers i.e., glycosides. Other useful nonionic surfactants include the condensation products of alkylene oxides with fatty acids (i.e. alkylene oxide esters of fatty acids). These materials have the general formula RCO(X)ₙOH wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 200. Other nonionic surfactants are the condensation products of alkylene oxides with 2 moles of fatty acids (i.e. alkylene oxide diesters of fatty acids). These materials have the general formula RCO(X)ₙOOCR wherein R is a C₁₀₋₃₀ alkyl group, X is —OCH₂CH₂-(i.e. derived from ethylene glycol or oxide) or —OCH₂CHCH₃-(i.e. derived from propylene glycol or oxide), and n is an integer from about 6 to about 100. Even further suitable examples include a mixture of cetearyl alcohols, cetearyl glucosides such as those available under the trade name Montanov 68 from Seppic and Emulgade PL68/50 from Cognis UK Ltd.. An example of a suitable cetearyl glucoside material without added fatty alcohols is Tego (RTM) Care CG90 commercially available from Goldschmidt GmbH.

The hydrophilic surfactants useful herein can alternatively or additionally include any of a wide variety of cationic, anionic, zwitterionic, and amphoteric surfactants such as are known in the art. See, e.g., McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973. A wide variety of anionic surfactants are also useful herein. See, e.g., U.S. Pat. No. 3,929,678, to Laughlin et al., issued Dec. 30, 1975.

A wide variety of anionic surfactants are also useful herein. See, e.g., U.S. Pat. No. 3,929,678, to Laughlin et al., issued Dec. 30, 1975. Exemplary anionic surfactants include the alkoyl isethionates (e.g., C₁₂-C₃₀), alkyl and alkyl ether sulfates and salts thereof, alkyl and alkyl ether phosphates and salts thereof, alkyl methyl taurates (e.g., C₁₂-C₃₀), and soaps (e.g., alkali metal salts, e.g., sodium or potassium salts) of fatty acids.

Amphoteric and zwitterionic surfactants are also useful herein. Examples of amphoteric and zwitterionic surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 22 carbon atoms (preferably C₈-C₁₈) and one contains an anionic water solubilising group, e.g., carboxy, sulfonate, sulfate, phosphate, or phosphonate. Examples are alkyl imino acetates, and iminodialkanoates and aminoalkanoates, imidazolinium and ammonium derivatives. Other suitable amphoteric and zwitterionic surfactants are those selected from the group consisting of betaines, sultaines, hydroxysultaines, and branched and unbranched alkanoyl sarcosinates, and mixtures thereof.

Thickening Agent (Including Thickeners and Gelling Agents)

The compositions of the present invention can also preferably comprise a thickening agent, more preferably from about 0.1% to about 5%, even more preferably from about 0.1% to about 3%, and most preferably from about 0.25% to about 2%, of a thickening agent.

Preferred compositions of the present invention include a thickening agent selected from carboxylic acid polymers, crosslinked polyacrylates, polyacrylamides, xanthan gum and mixtures thereof, more preferably selected polyacrylamide polymers, xanthan gum and mixtures thereof. Preferred polyacrylamides are predispersed in a water-immiscible solvent such as mineral oil and the like, containing a surfactant (HLB from about 7 to about 10) which helps to facilitate water dispersibility of the polyacrylamide. Most preferred for use herein is the non-ionic polymer under the CTFA designation: polyacrylamide and isoparaffin and laureth-7, available under the trade name Sepigel 305 from Seppic Corporation. Also useful are acrylic acid/ethyl acrylate copolymers and the carboxyvinyl polymers sold by the B. F. Goodrich Company under the trade mark of Carbopol resins. Suitable Carbopol resins are described in WO98/22085. All thickening agents discussed in application WO 00/24372 should be considered as suitable for use in the present invention.

Inorganic Sunscreens

In addition to the organic sunscreens compositions of the present invention can additionally comprise inorganic physical sunblocks. Nonlimiting examples of suitable physical sunblocks are described in CTFA International Cosmetic Ingredient Dictionary, 6^{th} Edition, 1995, pp. 1026-28 and 1103, Sayre, R. M. et al., “Physical Sunscreens”, J. Soc. Cosmet. Chem., vol 41, no 2, pp. 103-109 (1990). Preferred inorganic physical sunblocks are zinc oxide and titanium dioxide, and mixtures thereof.

When used, the physical sunblocks are present in an amount such that the present compositions are transparent on the skin (i.e., non-whitening), preferably less than or equal to about 5%. When titanium dioxide is used, it can have an anatase, rutile, or amorphous structure. Physical sunblock particles, e.g., titanium dioxide and zinc oxide, can be uncoated or coated with a variety of materials including but not limited to amino acids, aluminium compounds such as alumina, aluminium stearate, aluminium laurate, and the like; carboxylic acids and their salts e.g., stearic acid and its salts; phospholipids such as lecithin; organic silicone compounds; inorganic silicone compounds such as silica and silicates; and mixtures thereof. A preferred titanium dioxide is commercially available from Tayca (Japan) and is distributed by Tri-K Industries (Emerson, N.J.) under the MT micro-ionised series (e.g., MT 100SAS).

The compositions of the present invention preferably comprise from about 0.1% to about 10%, more preferably from about 0.1% to about 4%, and most preferably from about 0.5% to about 2.5%, by weight, of inorganic sunscreen.

Actives for Regulating Skin Condition

The compositions of the invention optionally comprise a safe and effective amount of an active for regulating skin condition including prophylactically and therapeutically regulating the skin condition. Prophylactically regulating skin condition includes delaying, minimising, and/or preventing visible and/or tactile discontinuities in the skin. Therapeutically regulating the skin condiments includes ameliorating e.g., diminishing, minimising, and/or effacing such discontinuities. Regulating the skin condition also involves improving the skin appearance and/or feel. Also included is regulating the signs of ageing which can involve prophylactically regulating and/or therapeutically regulating one or more of such signs e.g., fine lines, wrinkles, pores etc.

Ingredients that are known to be useful for regulating the skin condition are selected from Vitamin B₃ compounds, retinoids, and combinations thereof. As described for the humectants, the aforementioned compounds may, when used by themselves, give rise to a high level of tack, especially when used at the higher levels. It has been found, however, that this tacky feel can be offset by using the particulates of the present invention. The compositions of the present invention preferably comprise from about 0.1% to about 15%, more preferably from about 0.3% to about 10%, even more preferably from about 1 to about 5% of the active.

As used herein, “vitamin B₃ compound” means a compound having the formula:

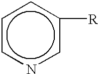

wherein R is —CONH₂ (i.e., niacinamide), —COOH (i.e., nicotinic acid) or —CH₂OH (i.e., nicotinyl alcohol); derivatives thereof; and salts of any of the foregoing. One or more vitamin B₃ compounds, or their salts, or mixtures thereof may be used herein. In a preferred embodiment, the vitamin B₃ compound typically contains less than about 50% of the compound in a salt form. As used herein, “retinoid” includes all natural and/or synthetic analogues of Vitamin A or retinol-like compounds which possess the biological activity of Vitamin A in the skin as well as the geometric isomers and stereoisomers of these compounds. Again, all skin regulating materials discussed in application WO 00/24372 should be considered as suitable for use in the present invention.

Anti-Inflammatory Agents

A safe and effective amount of an anti-inflammatory agent may be added to the compositions of the subject invention, preferably from about 0.1% to about 5%, more preferably from about 0.1% to about 2%, of the composition. The anti-inflammatory agent enhances the skin appearance benefits of the present invention, e.g., such agents contribute to a more uniform and acceptable skin tone or colour. The exact amount of anti-inflammatory agent to be used in the compositions will depend on the particular anti-inflammatory agent utilised since such agents vary widely in potency. Anti-inflammatory agents useful herein include steroids such as hydrocortisone; non-steroidal anti-inflammatory drugs (NSAIDS) such as ibuprofen; panthenol and ether and ester derivatives thereof e.g. panthenol ethyl ether, panthenyl triacetate; pantothenic acid and salt and ester derivatives thereof, especially calcium pantothenate; aloe vera, bisabolol, allantoin and compounds of the liquorice (the plant genus/species Glycyrrhiza glabra) family, including glycyrrhetic acid, glycyrrhizic acid, and derivatives thereof e.g. salts such as ammonium glycyrrhizinate and esters such as stearyl glycyrrhetinate. Particularly preferred herein are panthenol, pantothenic acid and their ether, ester or salt derivatives and mixtures thereof; suitable levels are from about 0.1 to about 5%, preferably from about 0.5 to about 3%. Panthenol is especially preferred.

Anti-Oxidants/Radical Scavengers

Compositions of the subject invention can further include an anti-oxidant/radical scavenger. The anti-oxidant/radical scavenger is especially useful for providing protection against UV radiation which can cause increased scaling or texture changes in the stratum corneum and against other environmental agents which can cause skin damage. Suitable amounts are from about 0.1% to about 10%, more preferably from about 1% to about 5%, of the composition.

Anti-Oxidants/Radical Scavengers

Anti-oxidants/radical scavengers such as ascorbic acid (vitamin C) and its salts, ascorbyl esters of fatty acids, ascorbic acid derivatives (e.g., magnesium ascorbyl phosphate), β-carotene, tocopherol (vitamin E), tocopherol sorbate, tocopherol acetate, other esters of tocopherol, butylated hydroxy benzoic acids and their salts, gallic acid and its alkyl esters, especially propyl gallate, uric acid and its salts and alkyl esters, sorbic acid and its salts, amines (e.g., N,N-diethylhydroxylamine, amino-guanidine), sulfhydryl compounds (e.g., glutathione), dihydroxy fumaric acid and its salts, bioflavonoids, lysine, methionine, proline, superoxide dismutase, silymarin, tea extracts, grape skin/seed extracts, melanin, and rosemary extracts may be used. Preferred anti-oxidants/radical scavengers are selected from tocopherol acetate, tocopherol sorbate and other esters of tocopherol, more preferably tocopherol acetate. As described for the humectants, the aforementioned compounds may, when used by themselves, give rise to a high level of tack, especially when used at the higher levels. It has been found, however, that this tacky feel can be offset by using the particulates of the present invention.

Chelators

The inclusion of a chelating agent is especially useful for providing protection against UV radiation which can contribute to excessive scaling or skin texture changes and against other environmental agents which can cause skin damage. A suitable amount is from about 0.01% to about 1%, more preferably from about 0.05% to about 0.5%, of the composition. Exemplary chelators that are useful herein are disclosed in U.S. Pat. No. 5,487,884, incorporated herein by reference. Preferred chelators useful in compositions of the subject invention are ethylenediamine tetraacetic acid (EDTA), furildioxime, and derivatives thereof.

Desquamation Agents/Exfoliants

A safe and effective amount of a desquamation agent may be added to the compositions of the subject invention, more preferably from about 0.1% to about 10%, even more preferably from about 0.2% to about 5%, also preferably from about 0.5% to about 4% of the composition. Desquamation agents enhance the skin appearance benefits of the present invention. For example, the desquamation agents tend to improve the texture of the skin (e.g., smoothness). A variety of desquamation agents are known in the art and are suitable for use herein, including organic hydroxy acids such as salicylic acid, glycolic acid, lactic acid, 5-octanoyl salicylic acid, hydroxyoctanoic acid, hydroxycaprylic acid, and lanolin fatty acids. One desquamation system that is suitable for use herein comprises sulphydryl compounds and zwitterionic surfactants and is described in WO 96/01101, incorporated herein by reference. Another desquamation system that is suitable for use herein comprises salicylic acid and zwitterionic surfactants and is described in WO 95/13048, incorporated herein by reference. Salicylic acid is preferred.

Skin Lightening Agents

The compositions of the present invention can also comprise a skin lightening agent. When used, the compositions preferably comprise from about 0.1% to about 10%, more preferably from about 0.2% to about 5%, also preferably from about 0.5% to about 2%, of a skin lightening agent. Suitable skin lightening agents include those known in the art, including kojic acid, arbutin, ascorbic acid and derivatives thereof, e.g., magnesium ascorbyl phosphate. Further skin lightening agents suitable for use herein also include those described in WO 95/34280 and WO 95/23780; each incorporated herein by reference.

Silicone Based Ingredients

The compositions of the present invention preferably also contain silicone based ingredients. Preferred examples are discussed below:

i) Silicone Based Emollients. Organopolysiloxane oils may be used as ingredients with emollient benefits in the present compositions. Suitable organopolysiloxane oils include volatile, non-volatile, or a mixture of volatile and non-volatile silicones. The term “non-volatile” as used in this context refers to those silicones that are liquid under ambient conditions and have a flash point (under one atmospheric of pressure) of or greater than about 100° C. The term “volatile” as used in this context refers to those silicone oils having a flash point of less than 100° C. Suitable organopolysiloxanes can be selected from a wide variety of silicones spanning a broad range of volatilities and viscosities. Non-volatile polysiloxanes are preferred. Suitable silicones are disclosed in U.S. Pat. No. 5,069,897, issued Dec. 3, 1991, which is incorporated by reference herein in its entirety.

Preferred for use herein are organopolysiloxanes selected from the group consisting of polyalkylsiloxanes, alkyl substituted dimethicones, dimethiconols, polyalkylaryl siloxanes, and mixtures thereof. More preferred for use herein are polyalkylsiloxanes and cyclomethicones. Preferred among the polyalkylsiloxanes are dimethicones for example DC200 available from Dow Corning and SF96 available from GE Silicone.

ii) Silicone Based Emulsifiers. Preferred emulsions of the present invention include a silicone containing emulsifier or surfactant. A wide variety of silicone emulsifiers are useful herein. These silicone emulsifiers are typically organically modified organopolysiloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C₂-C₃₀ pendant side chains. Still other useful dimethicone copolyols include materials having various cationic, anionic, amphoteric, and zwitterionic pendant moieties.

Preparation of Compositions

The present invention relates to cosmetic compositions with an aqueous continuous phase comprising organic particulate, greater than 8% humectant and organic sunscreen. Compositions are preferably an oil-in-water emulsion. These can be prepared according to procedures usually used in cosmetics and that are well known and understood by those skilled in the art with materials of similar phase partitioning can be added in any order.

Preferably the organic particulate is slowly and homogeneously premixed into a carrier oil prior to addition to the balance of the composition. This results in the organic particulate being able to more efficiently of set the negative tack properties of the skin benefit agents than if the particulate is added directly to the composition. Once formed the premix should be preferably added immediately to the balance of the composition to maintain the benefits. If the balance of the composition is an emulsion then it is preferable to prepare the emulsion prior to addition of the premix. This avoids disruption of the premix that could potentially occur as a result of the high shear step during emulsification.

Premix Composition

The premix preferably comprises from about 5% to about 90%, preferably from about 20% to about 80% and more preferably from about 40% to about 75%, by weight, of organic particulate. Preferably it has a viscosity of less than 500 mPa.s, preferably less than 2000 mPa.s which is measured using an airbearing Haake rheostress RS75 rheometer using a C35/2Ti cone and plate and 0.105 mm gap distance. The viscosity is measured at 31° C. (skin temperature) at a shear rate of 20s⁻¹. Additionally it is preferred that the premix forms, and remains stable within, a discrete phase within the balance of the composition such that it can be viewed as distinct droplets, with a diameter of greater than 0.1 μm, when viewed at a magnification of 40× using a standard light microscope. However, it is recognised that, dependent upon the other ingredients within the composition, there may be migration from one phase to another of several of the other ingredients once the composition is formed which may prevent the maintenance of the pre-mix as a truly discrete phase.

Carrier Oil

It is preferred that the carrier oil has a surface tension of less than that of the chosen organic particulate and a viscosity of less than about 350 mPa.s, preferably less than about 100 mPa.s and more preferably less than about 75 mPa.s. The viscosity of the carrier oil is also measured at 31° C. (skin temperature), in the same way as already described for the total premix.

The carrier oil preferably is a organosilicone based oil selected from non volatile and volatile organosiloxanes, and mixtures thereof. The term “non-volatile” as used in this context refers to those silicones that are liquid under ambient conditions and have a flash point (under one atmospheric of pressure) of or greater than about 100° C. The term “volatile” as used in this context refers to those silicone oils having a flash point of less than 100° C. Suitable organopolysiloxanes can be selected from a wide variety of silicones spanning a broad range of volatilities and viscosities. Non-volatile organopolysiloxanes are preferred. Suitable silicone carrier oils are disclosed in U.S. Pat. No. 5,069,897, issued Dec. 3, 1991. Preferred for use herein are organopolysiloxanes selected from the group consisting of polyalkylsiloxanes, alkyl substituted dimethicones, dimethiconols, polyalkylaryl siloxanes, and mixtures thereof. More preferred for use herein are polyalkylsiloxanes and cyclomethicones. Preferred among the polyalkylsiloxanes are dimethicones, for example DC200 available from Dow Corning and SF96, available from GE Silicone.

### EXAMPLES

The following examples further illustrate the preferred embodiments within the scope of the present invention. These examples are given solely for the purpose of illustration and are not to be construed as limitations of the present invention as many variations of the invention are possible without departing from its spirit or scope. Unless otherwise indicated, all ingredients are expressed as a weight percentage of the active ingredient.

Phase I

| EXAMPLE | I | II | III | IV | V | VI |
|---|---|---|---|---|---|---|
| Parsol HS¹ | 1.00 | 1.00 | 1.00 | — | — | 1.00 |
| Parsol 1789¹ | 1.00 | 1.50 | 2.00 | 2.00 | 2.00 | 1.00 |
| Octocrylene² | 1.00 | 2.00 | 1.50 | | | 1.00 |
| Octyl salicylate³ | 2.50 | 3.50 | 4.50 | 3.00 | | 2.50 |
| Parsol MCX¹ | — | — | — | — | 5.00 | — |
| Stearyl Alcohol | 0.40 | 0.40 | 0.48 | 0.80 | 1.2 | 0.40 |
| Cetyl Alcohol | 1.00 | 0.80 | 0.72 | 1.80 | 0.80 | 1.00 |
| Hydrofol Acid | 0.10 | 0.10 | 0.10 | 0.10 | 0.10 | 0.10 |
| Myrj 59⁴ | 0.10 | 0.10 | 0.10 | 0.10 | 0.10 | 0.10 |
| Isopropyl Isostearate⁵ | 0.75 | 1.00 | 1.00 | 0.5 | 2.00 | 0.75 |
| SEFA Cottonate⁶ | 1.50 | 1.00 | 1.80 | — | — | 1.50 |
| Ethyl paraben | 0.15 | 0.15 | 0.20 | 0.20 | 0.25 | 0.15 |
| Propyl paraben | 0.20 | 0.20 | 0.30 | 0.25 | 0.25 | 0.20 |
| Tocopherol acetate | 0.25 | 0.50 | 0.75 | 0.50 | — | 0.25 |
| Isohexadecane | — | 4.00 | — | 0.50 | 1.00 | — |
| Petrolatum | — | — | — | 1.20 | 0.50 | — |

Phase II

| EXAMPLE | I | II | III | IV | V | VI |
|---|---|---|---|---|---|---|
| Deionised Water | to | to | to | to | to | to |
| | 100 | 100 | 100 | 100 | 100 | 100 |
| Glycerine | 9.00 | 12.00 | — | 15.00 | — | 9.00 |
| Kobo GWL75CAP⁷ | 0.30 | 0.25 | 0.50 | 0.50 | 0.50 | 0.30 |
| Polyethylene | 1.00 | — | — | | 10.00 | 1.00 |
| glycol 200⁸ | | | | | | |
| Hexane-126-triol | — | 0.50 | 3.00 | — | — | — |
| Urea | — | — | 10.00 | — | — | — |
| Montanov 68⁹ | — | 0.50 | 0.20 | 0.50 | 0.20 | — |
| Emulgade | — | — | — | — | — | 0.25 |
| PL68/50¹⁰ | | | | | | |
| Di-sodium EDTA | 0.10 | 0.10 | 0.10 | 0.10 | 0.10 | 0.10 |

Phase III

| EXAMPLE | I | II | III | IV | V | VI |
|---|---|---|---|---|---|---|
| Fluoropure 100C¹¹ | 0.50 | — | — | — | — | — |
| Tospearl 145A¹² | — | 1.00 | — | — | 1.00 | 1.00 |
| Tospearl 120A¹² | — | — | — | 1.00 | — | — |
| Orgasol 2002 EXD N COS¹³ | — | — | 1.00 | — | — | — |
| DC200 5cs¹⁴ | — | 0.65 | 1.50 | — | — | — |
| DC200 50cs¹⁴ | 0.17 | — | — | 0.50 | — | — |
| DC200 100cs¹⁴ | — | — | — | 2.50 | — | — |
| Isohexadecane | 0.15 | — | 0.50 | — | — | — |

Additional Ingredients

| EXAMPLE | I | II | III | IV | V | VI |
|---|---|---|---|---|---|---|
| Sepigel 305⁹ | 1.50 | 2.00 | 3.00 | 2.00 | 3.00 | 1.50 |
| Sodium Hydroxide pellets | 0.013 | 0.013 | 0.013 | 0.013 | 0.013 | 0.013 |
| Benzyl alcohol | 0.20 | 0.25 | 0.25 | 0.25 | 0.30 | 0.20 |
| D-Panthenol | — | 0.50 | 0.50 | — | 1.00 | — |
| Niacinamide | — | 3.00 | — | 2.00 | 5.00 | — |
| DC Q2-1403¹⁴ | 1.60 | 2.00 | 1.50 | 2.00 | 2.50 | 1.60 |
| Dry Flo¹⁵ | — | — | — | — | — | 2 |
| ¹Supplied by Hoffmann-LA Roche, Inc., 340 Kingsland Street, Nutley, NJ 07110-1199 | | | | | | |
| ²Supplied by BASF Aktiengesellschaft, MEM/KU-D 205, 67056 Ludwigshafen, Germany | | | | | | |
| ³Supplied by Haarmann & Reimer Corporation, 70 Diamond Road, P.0. Box 175, Springfield, NJ 07081-0175 | | | | | | |
| ⁴PEG 100 Stearate supplied by 101, P0 Box 90, Wilton Centre, Middlesborough, Cleveland, England. TS6 8JE | | | | | | |
| ⁵Supplied by Scher Chemicals Inc, Industrial West, Clifton, NJ 07012 | | | | | | |
| ⁶A C1-C30 monoester or polyester of sugars and one or more carboxylic acid moieties as described herein, preferably a sucrose polyester in which the degree of esterification is 7-8, and in which the fatty acid moieties are C18 mono- and/or di-unsaturated and behenic, in a molar ratio of unsaturates:behenic of 1:7 to 3:5, more preferably the octaester of sucrose in which there are about 7 behenic fatty acid moieties and about 1 oleic acid moiety in the molecule, e.g., sucrose | | | | | | |
| # ester of cottonseed oil fatty acids. | | | | | | |

⁷Supplied by Kobo Products Inc, 690 Montrose Ave, So Plainfield, NJ 07080
⁸Supplied by Union Carbide Corporation, Danbury, Conn., USA
⁹Supplied by Seppic, 75 Quai D'Orsay, Paris
¹⁰Supplied by Cognis UK Ltd., 3^{rd} floor, Eleanor House, 33/35 Eleanor Cross Road, Waltham Cross, EN8 7LE, UK
¹¹Supplied by Shamrock Technologies Inc., Newark, NJ07114, USA
¹²Supplied by GE Silicones, Plasticslaan 1/PO Box 117, 4600 AC Bergen op Zoom, Netherlands
¹³Supplied by Elf Atochem, 4, cours Michelet, La Defense 10, Cedex 42, 92091, Paris
¹⁴Supplied by Dow Corning, Kings Court, 185 Kinds Rd, Reading, Berks, RG1 4EX
¹⁵Aluminium Starch Octenyl Succinate supplied by National Starch and Chemical Limited, Braunston, Daventry, Northants., NN11 7KL, UK

Phase I was prepared by mixing the oil soluble emulsifiers, emollients and oil soluble preservatives with gentle heating until an homogenous mixture was formed.

Phase II was prepared by heating water, glycerine, Kobo titanium dioxide, urea, polyethylene glycol, hexane-1, 2, 6-triol in a vessel to about 80° C. while mixing with a Heidolph pitch blade turbine mixer at 200 rpms, cooling to 75° C. and adding Montanov 68 or Emulgade PL68/50 and disodium EDTA. (Note that the Montanov 68 and Emulgade PL68/50 can be added into the oil phase instead of the water phase if desired). Stirring was continued for a further 5 minutes prior to addition of Phase I. The mixture of Phase I with Phase II was homogenised using an Ultraturax T25 (IKA—Labortechnik) at 13500 rpms for about 10 minutes and then cooled to 65° C.

If required the particulate of Phase III was stirred into the carrier oil at room temperature with an impeller stirrer at 50-150 rpms. Once an homogenous mixture was formed it was added immediately to the Phase I/Phase II emulsion. If no carrier oil was present in Phase III the particulate was added slowly, with stirring, to the Phase I/Phase II emulsion.

The resultant mixture was cooled further and once it reached 60° C. the Sepigel 305 and NaOH were added. The remainder of the ingredients were added after the mixture had been further cooled to 50° C. The resultant product was cooled further prior to packaging.

What is claimed is:

1. A topical cosmetic composition with an aqueous continuous phase, in the form of a lotion, cream or gel, comprising:
i) from about 0.01% to about 25%, by weight, of an organic particulate material having a median particle size of from about 0.5 to about 30 μm;
ii) greater than about 8%, by weight, of a humectant selected from the group consisting of glycerine, polyalkylene glycol, urea, D or DL panthenol, hexane-1, 2, 6-triol, guanidine or its derivatives, alkylene glycols such as butylene glycol and propylene glycol, and mixtures thereof;
iii) from about 2% to about 20%, by weight, organic sunscreen.

2. A composition according to claim 1 wherein the organic particulate material has a median particle size from about 0.5 to about 15 μm.

3. A composition according to claim 1 wherein the organic particulate material is selected from organosilicone polymers, polyethylene, polyacrylonitrile, polyacrylic acid, polymethacrylic acid, polystyrene, polytetrafluoroethylene, poly(vinylidene chloride), polyamide and ethylene/acrylic acid copolymer, and mixtures thereof.

4. A composition according to claim 3 wherein the organic particulate is polymethylsilsesquioxane.

5. A composition according to claim 1 wherein the organic particulate material has a critical surface tension of less than about 50 mN/m.

6. A composition according to claim 1 which comprises from about 0.1% to about 15%, by weight, organic particulate material.

7. A composition according to claim 1 wherein the humectant is selected from the group consisting of glycerine, urea and mixtures thereof.

8. A composition according to claim 1 which comprises from about 8% to about 30%, by weight, of humectant.

9. A composition according to claim 1 wherein the organic sunscreen is selected from the group consisting of compounds with UVA absorbing properties, compounds with UVB absorbing properties and mixtures thereof.

10. A composition according to claim 9 wherein the organic UVA absorbing sunscreen is a dibenzoylmethane derivative.

11. A composition according to claim 9 wherein the organic UVB absorbing sunscreen is selected from the group consisting of octocrylene, octomethoxy cinnamate, octyl salicylcate, phenyl benzimidazol sulphonic acid and mixtures thereof.

12. A composition according to claim 1 which comprises from about 4% to about 14%, by weight, of organic sunscreen.

13. A composition according to claim 12 wherein the Sun Protection Factor is greater than 10, and the critical absorption wave length is greater than 370 nm.

14. A composition according to claim 1 comprising from about 0.1% to about 20%, by weight, of niacinamide.

15. A composition according to claim 1 which is an oil-in-water emulsion.

16. A composition according to claim 15 whereby the particulate material is pre-dispersed in an organosiloxane oil and whereby said premix has a viscosity of less than about 5000 cps at 31° C. at 20s⁻¹.

17. A composition of claim 16 wherein the organosiloxane oil dispersion fluid has a viscosity less than about 350 mPa.s at 31° C. at 20s⁻¹.

18. Use of a composition according to claim 17 wherein said organosiloxane oil is dimethicone.

19. A method for delivering a high level of moisturisation and UV protection to the skin with low levels of tackiness or stickiness by application of the composition of claim 1.
